(19) United States
(12) Patent Application Publication
(10) Pub. No.: US 2007/0010648 A1
(43) Pub. Date: Jan. 11, 2007

(54) Catalyst for manufacture of esters

(76) Inventors: Martin Graham Partridge, Cambridgeshire (GB); Calum Harry McIntosh, Cleveland (GB); Alan Joseph Hanratty, Cleveland (GB)

Correspondence Address: RATNERPRESTIA, P O BOX 980, VALLEY FORGE, PA, 19482-0980, US

(21) Appl. No.: 10/574,976

(22) PCT Filed: Oct. 5, 2004
(86) PCT No.: PCT/GB04/04218

§ 371 Date: Apr. 7, 2006

(30) Foreign Application Priority Data
Oct. 7, 2003 (GB) 0323386.3

(51) Int. Cl. B01J 31/00; C07C 69/76; C08G 63/00

(52) U.S. Cl. 528/274; 502/150; 502/162; 502/167; 502/152; 560/89

(57) ABSTRACT

A catalyst suitable for use in an esterification reaction comprises the reaction product of a compound of titanium, zirconium or hafnium, a 2hydroxy carboxylic acid and a quaternary ammonium compound selected from the group consisting of tetraethylammonium hydroxide and tetramethylammonium hydroxide, optionally with an alcohol or water.

The present invention relates to a catalyst composition which is particularly useful in the manufacture of esters, especially polyesters, and to manufacturing processes using the catalyst composition and also to ester products containing residues of the catalyst composition.

Certain metals and metal-containing compositions are well known for use to catalyse ester-forming reactions, including esterification and transesterification. Titanium compounds such as titanium alkoxides may be used in the manufacture of polyesters in addition to or in place of other metal compounds such as antimony compounds. Antimony compounds are very commonly used catalysts for polyester manufacture but have certain disadvantages, which include the inherent toxicity of antimony and also the fact that antimony residues may remain in the polyester, giving a grey colour or, in extreme cases, small visible particles in the polyester. Therefore titanium catalysts, which are highly active esterification catalysts, provide attractive alternatives to antimony in polyester manufacture in order to reduce or eliminate the requirement for antimony compounds. Titanium catalysts, however, have the disadvantage that the titanium compounds remaining in the polymer tend to produce a yellow colouration. If the final use of the polyester product requires a neutral-coloured or “water-white” material, the colour of the polyester may be adjusted by adding blueing compounds or toners. Inorganic toners such as cobalt acetate are common although a desire to reduce the cobalt content of the polyester has prompted an increase in the use of organic dyes to counteract the yellow colour imparted by titanium catalysts. The need for colour-management of the polyester by the addition of dyes or toners is inconvenient and adds to the costs of polyester production therefore it is desirable to reduce or avoid the need to use toners or other colour management additives.

It is therefore an object of the invention to provide- an improved catalyst composition for use in the production of esters. It is a further object of the invention to provide a catalyst composition which may be used in the production of polyesters and which produces a polyester of reduced yellow colouration compared with known titanium-based catalyst compositions.

In EP-A-0812818, a process for the preparation of an ester comprises carrying out an esterification in the presence of a catalyst comprising the reaction product of an orthoester or a condensed orthoester of titanium or zirconium, an alcohol containing at least two hydroxyl groups, a 2-hydroxy acid and a base. These catalysts are more stable than simple titanium alkoxide catalysts and are useful in producing polyester of better colour. There is, however, no suggestion that selection of certain compounds as bases may produce an improved catalyst of the present invention.

WO01/56694 discloses a catalyst composition suitable for use as a catalyst for the preparation of an ester, including a polyester, comprising an organometallic compound which is a complex of first metal selected from the group consisting of titanium and zirconium, a second metal selected from the group consisting of germanium, antimony and tin and a carboxylic acid, preferably in the presence of an alcohol having at least two hydroxy groups and a base. Although these bi-metallic complexes contain a base, there is no disclosure that selection of an organic base would lead to any particular advantage over the preferred inorganic bases.

WO02/42537 discloses that a combination of a catalyst of the type disclosed in EP-A-0812818 with a second catalyst component selected from a compound of antimony, germanium or tin, is particularly effective in the manufacture of polyester for fibre spinning applications. Although quaternary ammonium compounds, are mentioned as suitable bases, there is no disclosure that the catalyst compositions of the present invention are particularly effective in producing a polyester having reduced yellowness in the absence of antimony, germanium or tin.

According to the invention, we provide a catalyst suitable for use in an esterification reaction comprising the reaction product of
a) a compound of titanium, zirconium or hafnium
b) a 2-hydroxy carboxylic acid and
c) a quaternary ammonium compound selected from the group consisting of tetraethylammonium hydroxide and tetramethylammonium hydroxide.

According to a second aspect of the invention, we provide a process for the production of an ester, including a polyester, comprising reacting together an alcohol, which may be a polyhydroxy alcohol and at least one carboxylic acid, which may be a multifunctional carboxylic acid, or an ester thereof to form an ester, which may be a polyester, said reaction taking place in the presence of a catalyst according to the invention. Preferably the reaction is carried out in the absence of a catalytically effective quantity of antimony, germanium or tin.

According to a third aspect of the invention, we provide a process for the production of a polyester comprising:
a) reacting together a polyhydroxy alcohol with at least one multifunctional carboxylic acid or an ester thereof to form a polyhydroxy ester of the multifunctional carboxylic acid,
b) polycondensing said polyhydroxy ester to form a polyester,
   characterised in that at least one of steps a) and b) is carried out in the presence of a catalyst according to the invention and preferably in the absence of a catalytically effective quantity of antimony, germanium or tin.

The compound of titanium, zirconium or hafnium is preferably an alkoxide or condensed alkoxide. Such alkoxides have the formula M(OR)₄ in which M is titanium, zirconium or hafnium and R is an alkyl group. More preferably R contains 1 to 6 carbon atoms and particularly suitable alkoxides include tetraisopropoxy titanium, tetra-n-butoxy titanium, tetra-n-propoxy zirconium and tetra-n-butoxy zirconium. The compound of titanium, zirconium or hafnium is preferably a compound of titanium. The condensed alkoxides suitable for preparing the catalysts useful in this invention are typically prepared by careful hydrolysis of titanium or zirconium alkoxides and are frequently represented by the formula R¹O[M(OR¹)₂O]ₙR¹ in which R¹ represents an alkyl group and M represents titanium or zirconium. Preferably, n is less than 20 and more preferably is less than 10. Preferably R¹ contains 1 to 6 carbon atoms and useful condensed alkoxides include the compounds known as polybutyl titanate, polyisopropyl titanate and polybutyl zirconate.

Preferred 2-hydroxy carboxylic acids include lactic acid, citric acid, malic acid and tartaric acid. Some suitable acids are supplied as hydrates or as aqueous solutions. Acids in this form as well as anhydrous acids are suitable for preparing the catalysts used in this invention. The preferred molar ratio of 2-hydroxy carboxylic acid to titanium, zirconium or hafnium in the reaction product is 1 to 4 moles per mole of titanium, zirconium or hafnium. More preferably the catalyst contains 1.5 to 3.5 moles of 2-hydroxy acid per mole of titanium, zirconium or hafnium.

The molar ratio of quaternary ammonium compound to 2-hydroxy carboxylic acid is preferably in the range 0.05 to 2:1. In the case of citric acid (a tribasic acid), the preferred amount is in the range 0.1 to 1.5 moles quaternary ammonium compound per mole of 2-hydroxy acid. In general, the amount of quaternary ammonium compound present is usually in the range 0.05 to 4 moles per mole of titanium, zirconium or hafnium and preferably the amount of quaternary ammonium compound is from 2 to 3 moles per mole of titanium, zirconium or hafnium. It is frequently convenient to add water together with the quaternary ammonium compound when preparing the catalysts, because the quaternary ammonium compounds are soluble in water and conveniently used as aqueous solutions.

The catalyst may, optionally, contain an alcohol, preferably an alcohol containing more than one hydroxyl group. Preferably the alcohol is a dihydric alcohol e.g.1,2-ethanediol, 1,2-propanediol, 1,3-propanediol, 1,4-butane diol or a dihydric alcohol containing a longer chain such as diethylene glycol or a polyethylene glycol. Particularly preferred is 1,2-ethanediol or 1,4-butane diol. The catalyst can also be prepared from a higher polyhydric alcohol such as glycerol, trimethylolpropane or pentaerythritol or a mono alcohol such as an aliphatic, cyclo-aliphatic or aromatic alcohol, e.g. a C₁-C₂₂ alcohol, e.g. ethanol, methanol, pentanol, butanol, isopropanol, cyclohexanol, 2-ethylhexanol, octanol etc. When the catalyst is intended for polyester manufacture, the added alcohol preferably contains at least two hydroxyl groups and is preferably of a similar composition to that used in the polyester manufacture. The alcohol, if present may be added to the catalyst reaction mixture at any stage including after the reaction of the metal compound with the 2-hydroxyacid and the quaternary ammonium compound. The prepared catalyst may be diluted in a further quantity of the alcohol. Water may be added to the reaction mixture during or after the preparation of the catalyst, and may be present as a solvent for the 2-hydroxyacid or the quaternary ammonium compound.

In a preferred form, therefore, especially useful as a catalyst for use in polyester manufacture, the invention comprises a catalyst comprising the reaction product of
a) a compound of titanium, zirconium or hafnium
b) an alcohol containing at least two hydroxyl groups,
c) a 2-hydroxy carboxylic acid and
d) a quaternary ammonium compound selected from the group consisting of tetraethylammonium hydroxide and tetramethylammonium, hydroxide.

Preferably this catalyst comprises from 2 to 12 moles of dihydric alcohol to each mole of the titanium, zirconium or hafnium. More preferably the catalyst contains from 3 to 8 moles dihydric alcohol per mole of titanium, zirconium or hafnium. A further quantity of alcohol or water may be added to the catalyst.

The catalyst can be prepared by mixing the components (metal compound, alcohol (if used), 2-hydroxy acid and quaternary ammonium compound) with removal of any by-product, (e.g. isopropyl alcohol when the metal compound is an alkoxide such as tetraisopropoxytitanium), at any appropriate stage. In one preferred method a metal alkoxide or condensed alkoxide and dihydric alcohol are mixed and subsequently, 2-hydroxy acid and then quaternary ammonium compound are added or a pre-neutralised 2-hydroxy acid solution, is added. In an alternative preferred method a metal alkoxide or condensed alkoxide is first reacted with the 2-hydroxy acid. By-product alcohol may optionally be removed at this stage. Quaternary ammonium compound is then added to this mixture, to produce the reaction product which is a catalyst of the invention, optionally followed by dilution with an alcohol and/or water. If desired, by-product alcohol can be removed, e.g. by distillation, at any stage of the preparation process, e.g. before or after the dilution of the preferred product with a dihydric alcohol. When the components of the reaction mixture, especially the 2-hydroxyacid and the quaternary ammonium compound, are added as aqueous solutions, the reaction mixture contains water, which may be removed by distillation, optionally together with the by-product alcohol from the metal alkoxide, if used. The catalyst may be diluted in a solvent, which is preferably the alcohol to be used in the esterification reaction but which may comprise another solvent such as a different alcohol or water. For example, if the catalyst is to be used for making polyethylene terephthalate, then the catalyst may be diluted in 1,2-ethanediol.

The esterification reaction of the process of the invention can be any reaction by which an ester is produced. The reaction maybe a direct esterification in which a carboxylic acid or its anhydride react with an alcohol to form an ester; or a transesterification (alcoholysis) in which a first alcohol reacts with a first ester to produce an ester of the first alcohol and a second alcohol produced by cleavage of the first ester; or a interesterification reaction in which two esters are reacted to form two different esters by exchange of alkoxy radicals.

Many carboxylic acids and anhydrides can be used in direct esterification including saturated and unsaturated monocarboxylic acids such as stearic acid, isostearic acid, capric acid, caproic acid, palmitic acid, oleic acid, palmitoleic acid, triacontanoic acid, benzoic acid, methyl benzoic acid and salicylic acid, dicarboxylic acids such as phthalic acid, isophthalic acid, terephthalic acid, sebacic acid, adipic acid, azelaic acid, succinic acid, fumaric acid, maleic acid, naphthalene dicarboxylic acid and pamoic acid and anhydrides of these acids and polycarboxylic acids such as trimellitic acid, citric acid, trimesic acid, pyromellitic acid and anhydrides of these acids. Alcohols frequently used for direct esterification include aliphatic straight chain and branched monohydric alcohols such as butyl, pentyl, hexyl, octyl and stearyl alcohols and polyhydric alcohols such as glycerol and pentaerythritol. A preferred process of the invention comprises reacting 2-ethylhexanol with phthalic anhydride to form bis(2-ethylhexyl)phthalate.

The esters employed in an alcoholysis reaction are generally the lower homologues such as methyl, ethyl and propyl esters since, during the esterification reaction, it is usual to eliminate the displaced alcohol by distillation. Such esters of the acids suitable for direct esterification are used in the process of the invention. Frequently (meth)acrylate esters of longer chain alcohols are produced by alcoholysis of esters such a methyl acrylate, methyl methacrylate, ethyl acrylate and ethyl methacrylate. Typical alcohols used in alcoholysis reactions include butyl, hexyl, n-octyl and 2-ethyl hexyl alcohols and substituted alcohols such as dimethylaminoethanol. When the esterification reaction is a transesterification between two esters, generally the esters will be selected so as to produce a volatile product ester which can be removed by distillation.

Polymeric esters can be produced by processes involving direct esterification or transesterification and a particularly preferred embodiment of the process of the invention is a polyesterification reaction in the presence of the catalyst described hereinbefore. In a polyesterification reaction polybasic acids or esters of polybasic acids are usually reacted with polyhydric alcohols to produce a polymeric ester, often via a diester intermediate product. Typical polyacids used in polyester manufacture include terephthalic acid, isophthalic acid, naphthalene dicarboxylic acid (especially 2,6,-naphthalene dicarboxylicacid) and substituted versions of these acids, e.g. acids containing a sulphonate group. Aliphatic polyacids may also be used, particularly C₄-C₁₀ aliphatic dicarboxylic acids. Alternatively, the preparation of polyesters may be achieved starting from an ester (typically a low alkyl ester) of a dicarboxylic acid, which may be e.g. a C₁-C₆ alkyl ester of any of the di- or poly-carboxylic acids mentioned above. Of these, methyl esters such as, in particular dimethyl terephthalate or dimethyl naphthalate, are preferred starting materials for the preparation of polyesters. Preferred polyesterification reactions according to the invention include the reaction of terephthalic acid or dimethyl terephthalate with 1,2-ethanediol (ethylene glycol) to produce polyethylene terephthalate (PET), with 1,3-propane diol to form polypropylene terephthalate (also known as poly(trimethylene)terephthalate or PTT), or with 1,4-butanediol (butylene glycol) to produce polybutylene terephthalate (PBT) or reaction of naphthalene dicarboxylic acid with 1,2-ethanediol to produce polyethylene naphthalate (PEN). Other glycols or higher polyols such as 1,6-hexanediol, bishydroxymethylene-cyclohexane (cyclohexane dimethanol), pentaerythritol and similar diols are also suitable for preparing polyesters and may be used in mixtures to produce co-polyesters.

The catalyst and process of the present invention are particularly suitable for the preparation of PET, PBT, or PTT by the reaction of terephthalic acid or an ester thereof with 1,2-ethanediol, 1,4-butane diol, or 1,3-propane diol. We have found that the catalyst and process of the invention show numerous benefits compared with the known titanium alkoxide catalysts.

A typical process for the preparation of a polyester such as polyethylene terephthalate comprises two stages. In the first stage dimethyl terephthalate or terephthalic acid is reacted with 1,2-ethanediol to form a prepolymer and the by-product methanol or water is removed. The prepolymer is subsequently heated in a second stage under reduced pressure to remove 1,2-ethanediol and form a long chain polymer. Either or both these stages may comprise an esterification process according to this invention. A typical process for the preparation of polybutylene terephthalate is similar although in the first stage dimethyl terephthalate is normally used and the dihydric alcohol used is 1,4-butanediol. Processes may be operated either on a batch or a continuous basis. A preferred means of adding the catalyst compositions of this invention to a polyesterification reaction is in the form of a solution in the glycol being used (e.g. ethylene glycol in the preparation of polyethylene terephthalate). This method of addition is applicable to addition of the catalyst composition to the polyesterification reaction at the first stage or at the second stage.

The esterification reaction of the invention can be carried out using any appropriate, known technique for an esterification reaction.

In direct-esterification the acid or anhydride and an excess of alcohol are typically heated, if necessary in a solvent, in the presence of the catalyst. Water is usually the by-product of the reaction and this is removed, as an azeotrope with a boiling mixture of solvent and/or alcohol. Generally, the solvent and/or alcohol mixture which is condensed is immiscible with water which is therefore separated before solvent and/or alcohol are returned to the reaction vessel. When reaction is-complete the excess alcohol and, when used, solvent are evaporated. In contrast to prior art esterification processes, it is not generally necessary to remove the catalyst from the reaction mixture. A typical direct esterification reaction is the preparation of bis(2-ethylhexyl) phthalate which is prepared by mixing phthalic anhydride and 2-ethyl hexanol. An initial reaction to form a monoester is fast but the subsequent conversion of the monoester to diester is carried out by refluxing in the presence of the catalyst at a temperature of 180-200° C. until all the water has been removed. Subsequently,the excess alcohol is removed.

In an alcoholysis reaction, the ester, first alcohol and catalyst are mixed and, generally, the product alcohol (second alcohol) is removed by distillation often as an azetrope with the ester. Frequently it is necessary to fractionate the vapour mixture produced from the alcoholysis in order to ensure that the second alcohol is separated effectively without significant loss of product ester or first alcohol. The conditions under which alcoholysis reactions are carried out depend principally upon the components of the reaction and generally components are heated to the boiling point of the mixture used.

A preferred process of the invention is the preparation of polyethylene terephthalate. A typical batch production of polyethylene terephthalate is carried out by charging terephthalic acid and ethylene glycol to a reactor along with catalyst composition, if desired, and heating the contents to 260-270° C. under a pressure of about 0.3 Mpa (40 psi). Reaction commences as the acid dissolves and water is removed to form a bishydroxyethylterephthalate (BHET). Alternatively an ester such as dimethylterephthalate is used instead of the terephthalic acid and methanol is removed from the first stage of the reaction to form a bishydroxyethylterephthalate. The product is transferred to a second autoclave reactor and catalyst composition is added, if needed. The reactor is heated to 260-310° C. under an eventual vacuum of 100 Pa (1 mbar) to effect polycondensation. The molten product ester is discharged from the reactor, cooled and chipped. The chipped polyester may be then subjected to solid state polymerisation, if a higher molecular weight polymer is required. Typically, additives such as stabilisers (usually based on phosphorus compounds such as phosphoric acid and organic phosphates), colour toning compounds (such as cobalt compounds or organic dyes), pigments, etc are added to the reaction mixture during the melt polymerisation or at the first, esterification or transesterification stage.

A second preferred process of the invention is the preparation of polybutylene terephthalate. A typical batch production of polybutylene terephthalate is carried out by charging terephthalic acid and 1,4 butanediol to a reactor along with catalyst if desired and heating the contents to 170-210° C. under a pressure of about 0.3 MPa. Reaction commences as the acid dissolves at about 230° C. and water is removed. The product is transferred to a second autoclave reactor and catalyst is added, if needed. The reactor is heated to 240-260° C. under an eventual vacuum of 100 Pa to remove 1,4 butanediol by-product. The molten product ester is discharged from the reactor, cooled and chipped.

Conventional additives to polyesterification reactions, such as colour modifiers (e.g. cobalt compounds, pigments or dyes), stabilisers (especially those based on phosphorus compounds e.g. phosphoric acid or phosphate ester species), fillers etc may also be added to the polyester reaction mixture. Normally a phosphorus-containing stabiliser is added at a level of about 5-250 ppm P, especially 5-100 ppm, based upon product polyester.

The amount of catalyst used in the process of the invention generally depends upon the titanium or zirconium content, expressed as Ti or Zr, of the catalyst. Usually the amount is from 1 to 1000 parts per million (ppm) on weight of product ester for direct or transesterification reactions. Preferably the amount is from 2 to 450 ppm on weight of product ester and more preferably 5 to 50 ppm on weight of product ester. In polyesterification reactions the amount used is generally expressed as a proportion of the weight of product polyester and is usually from 2 to 500 ppm expressed as Ti or Zr based on product polyester. Preferably the amount is from 2 to 150 ppm expressed as Ti or Zr, more preferably from 2 to 50 ppm.

The catalyst of the invention may be used alone or in combination with known catalyst systems. In particular, for polyester manufacture which is normally carried out in two stages, it may be desirable to use an alternative catalyst for either the first (direct esterification or transesterification) stage or the second stage, the catalyst of the invention being used in the other stage. In some polyester processes no catalyst is used in the first stage of reaction to form BHET, the catalyst of the invention being used only for the polycondensation reaction. Optionally an additional catalyst may be used together with the catalyst of the invention in esterification or polyesterification (in either the first or the second stage of the polyester manufacturing process). Suitable co-catalysts in polyester manufacture include known antimony, magnesium, zinc, tin an d germanium catalysts.

In particular we have found that a combination of the catalyst of the invention with a zinc-containing compound is particularly beneficial in polyester manufacture. It has been found that the presence of a zinc compound provides an unexpected increase in the rate of melt polymerisation, enabling lower reaction temperatures to be used, and also a higher solid-phase polymerisation (SPP) rate compared with a catalyst system of the invention to which a zinc compound has not been added. Preferred zinc compounds are soluble in the polyester reaction medium and salts such as zinc acetate are particularly preferred. Zinc acetate is a well-known catalyst for use in polyester manufacture, however Its synergy with the catalysts of the invention to increase the SPP rate is unexpected. When a zinc compound is used for promotion of the rate of SPP, it is preferably present at a concentration of 5-200 ppm based on the amount of Zn in the final polyester composition.

The process of this invention has been shown to be effective for producing esters and polyesters at an economical rate.

The invention is illustrated by the following examples.

### EXAMPLE 1

### 3 Moles TEAH

A 50% w/w aqueous citric acid solution (959 g, 2.5 moles citric acid) was put in a flask. To the stirred solution was slowly added titanium isopropoxide (284 g, 1 mole) (VERTEG™ TIPT) and 100 g (1.6 moles) of isopropanol (IPA). This mixture was heated to 90° C. under reflux for 1 hour to yield a hazy solution and then distilled under vacuum to remove free water and isopropanol (300 g). The product was cooled below 50° C. and 35% w/w aqueous tetraethyl ammonium hydroxide (TEAH) (1262 g, 3 moles) was added slowly to the stirred solution followed by 496 g (8 moles) of ethylene glycol and heated under vacuum to remove free waterlisopropanol (1178 g). A further quantity of water (34 g) and ethylene glycol (631 g) was added to the product which was then refluxed at 90° C. for 60 minutes. The resulting product catalyst composition contained 2.1% Ti.

### EXAMPLE 2

### 2 Moles TEAH

A 50% w/w aqueous citric acid solution (480 g, 1.25 moles citric acid) was put in a flask. To the stirred solution was slowly added titanium Isopropoxide (142 g, 0.5 mole) and 50 g (0.8 moles) of isopropanol. This mixture was heated to 90° C. under reflux for 1 hour to yield a hazy solution and then distilled under vacuum to remove free water and isopropanol (151 g). The product was cooled below 50° C. and 35% w/w aqueous TEAH (421 g, 1 mole) was added slowly to the stirred solution followed by 248 g (4 moles) of ethylene glycol and heated under vacuum to remove free water/isopropanol (378 g). A further quantity of water (17 g) and ethylene glycol (315 g ) was added to the product which was then refluxed at 90° C. for 60 minutes. The resulting product catalyst composition contained 2.1% Ti.

### EXAMPLE 3

### 1 Mole TEAH

A 50% w/w aqueous citric acid solution (480 g, 1.25 moles citric acid) was put in a flask. To the stirred solution was slowly added titanium isopropoxide (142 g, 0.5 mole) and 50 g (0.8 moles) of isopropanol. This mixture was heated to 90° C under reflux for 1 hour to yield a hazy solution and then distilled under vacuum to remove free water and isopropanol (151 g). The product was cooled below 50° C. and 35% w/w aqueous TEAH (210 g, 0.5 moles) was added slowly to the stirred solution followed by 248 g (4 moles) of ethylene glycol and heated under vacuum to remove free water/isopropanol (168 g). A further quantity of water (1 7g) and ethylene glycol (315 g) was added to the product which was then refluxed at 90° C. for 60 minutes. The resulting product catalyst composition contained 2.1% Ti.

### EXAMPLE 4

### 3 Moles TEAH

A 50% w/w aqueous citric acid solution (960 g, 2.5 moles citric acid) was put in a flask. Titanium isopropoxide (284 g, 1 mole) (VERTEC™ TIPT) was added over a 20 minute period, followed by 50 g (0.8 moles) of isopropanol (IPA). This mixture was heated to 90° C. under reflux for 1 hour. The product was cooled and 35% w/w aqueous TEAH (1262 g, 3 moles) and 400 g water was added slowly to the stirred solution and heated under vacuum to remove free water/isopropanol. The resulting solid product catalyst composition contained 4.95% Ti.

### EXAMPLE 5

### 3 Mole TMAH

A 50% w/w aqueous citric acid solution (240 g, 0.62 moles citric acid) was put in a flask. To the stirred solution was slowly added titanium isopropoxide (71 g, 0.25 mole) and 25 g (0.42 moles) of isopropanol. This mixture was heated to 90° C. under reflux for 1 hour to yield a hazy solution and then distilled under vacuum to remove free water and isopropanol (74 g). The product was cooled below 50° C. and 25% w/w aqueous tetramethyl ammonium hydroxide (TMAH) (274 g, 0.75 moles) was added slowly to the stirred solution followed by 124 g (2 moles) of ethylene glycol and heated under vacuum to remove free water/isopropanol (253 g). A further quantity of water (9 g) and ethylene glycol (158 g) was added to the product which was then refluxed at 90° C. for 60 minutes. The resulting product catalyst composition contained 2.1% Ti.

### EXAMPLE 6

### 2 Mole TMAH: Mole Ti

A 50% w/w aqueous citric acid solution (240 g, 0.62 moles citric acid) was put in a flask. To the stirred solution was slowly added titanium isopropoxide (71 g, 0.25 mole) and 25 g (0.42 moles) of isopropanol. This mixture was heated to 90° C. under reflux for 1 hour to yield a hazy solution and then distilled under vacuum to remove free water and isopropanol (75 g). The product was cooled below 50° C. and 25% w/w aqueous TMAH (182 g, 0.50 moles) was added slovvly to the stirred solution followed by 124 g (2 moles) of ethylene glycol and heated under vacuum to remove free water/isopropanol (161 g). A further quantity of water (9 g) and ethylene glycol (158 g ) was added to the product which was then refluxed at 90° C. for 60 minutes. The resulting product catalyst composition contained 2.1% Ti.

### EXAMPLE 7

### 1 Mole TMAH

A 50% w/w aqueous citric acid solution (240 g, 0.62 moles citric acid) was put in a flask. To the stirred solution was slowly added titanium isopropoxide (71 g, 0.25 mole) and 25 g (0.42 moles) of isopropanol. This mixture was heated to 90° C. under reflux for 1 hour to yield a hazy solution and then distilled under vacuum to remove free water and isopropanol (75 g). The product was cooled below 50° C. and 25% w/w aqueous TMAH (91 g, 0.25 moles) was added slowly to the stirred solution followed by 124 g (2 moles) of ethylene glycol and heated under vacuum to remove free water/isopropanol (70 g). A further quantity of water (9 g) and ethylene glycol (158 g) was added to the product which was then refluxed at 90° C. for 60 minutes. The resulting product catalyst composition contained 2.1% Ti.

### EXAMPLE 8 (COMPARATIVE)

### 3 Moles Choline

A 50% w/w aqueous citric acid solution (480 g, 1.25 moles citric acid) was put in a flask. To the stirred solution was slowly added titanium isopropoxide (142 g, 0.5 mole) and 10 g (0.16 moles) of isopropanol. This mixture was heated to 90° C. under reflux for 1 hour to yield a hazy solution and then distilled under vacuum to remove free water and isopropanol (112 g). The product was cooled below 50° C. and 45% w/w aqueous choline hydroxide (403 g, 1.5 moles) was added slowly to the stirred solution followed by 284 g (4.5 moles) of ethylene glycol and heated under vacuum to remove free water/isopropanol (342 g). A further quantity of water (27 g) and ethylene glycol (286 g) was added to the product which was then refluxed at 90° C. for 60 minutes. The resulting product catalyst composition contained 2.1% Ti.

### EXAMPLE 9

### 3 Mole NH₄OH

A 50% w/w aqueous citric acid solution (480 g, 1.25 moles citric acid) was put in a flask. To the stirred solution was slowly added titanium isopropoxide (142 g, 0.50 mole) and 10 g (0.17 moles) of isopropanol. This mixture was heated to 90° C. under reflux for 1 hour to yield a hazy solution and then distilled under vacuum to remove free water and isopropanol (112 g). The product was cooled below 50° C. and 28% w/w aqueous ammonium hydroxide (188 g, 0.50 moles) was added slowly to the stirred solution followed by 248 g (4 moles) of ethylene glycol and heated under vacuum to remove free water/isopropanol 363 g. A further quantity of water (46 g) and ethylene glycol (503 g ) was added to the product which was then refluxed at 90° C. for 60 minutes. The resulting product catalyst composition contained 2.1% Ti.

### EXAMPLE 10 (COMPARATIVE)

The procedure of Example 1 was followed but using 132.5 g, (0.63 moles) of citric acid, 72.0 g, (0.25 moles) of titanium isopropoxide, 94.9 g, (0.76 moles) of 32% w/w aqueous sodium hydroxide and 125.5 g, (2.0 moles) of ethylene glycol. The product was a slightly hazy, very pale yellow liquid (Ti content 3.85% by weight).

### EXAMPLE 11 (COMPARATIVE)

The procedure of Example 1 was followed but using 132.5 g, (0.63 moles) of citric acid, 72.0 g, (0.25 moles) of titanium isopropoxide, 31 g, (0.25 moles) of 32% w/w aqueous sodium hydroxide and 125.5 g, (2.0 moles) of ethylene glycol. The product was a slightly hazy, very pale yellow liquid (Ti content 3.85% by weight).

### EXAMPLE 12

### Preparation of Poly(ethylene terephthalate) (PET)

Ethylene glycol (2.04 kg), isophthalic acid (125 g) and terephthalic acid (4.42 kg) were charged to a stirred, jacketed reactor. The catalyst was added and the reactor heated to 226-252° C. at a pressure of 40 psi to initiate the first stage direct esterification (DE) process. Water was removed 40 as it was formed with recirculation of the ethylene glycol. On completion of the DE reaction the contents of the reactor were allowed to reach atmospheric pressure before a vacuum was steadily applied. The mixture was heated to 290±2° C. under vacuum to remove ethylene glycol and yield polyethylene terephthalate. The final polyester was discharged once a constant torque had been reached which indicated an IV of about 0.62. The catalysts were added to produce a Ti content of 8 ppm in the polyester reaction mixture. The time for polycondensation (PC) and the intrinsic viscosity (IV) and colour values of the resulting polyesters are shown in Table 1. No inorganic or organic toners were added to the polymer. The colour of the polymer was measured using a Byk-Gardner Colourview spectrophotometer. A common model to use for colour expression is the Cielab L*, a* and b* scale where the b*-value describes yellowness. The yellowness of the polymer increases with b*-value.

The intrinsic viscosity (IV) was measured by solution viscosity on an 8% solution of the polyester in o-chlorophenol at 25° C.

TABLE 1

| | | PC | 5 minutes | | | 15 minutes | | |
|---|---|---|---|---|---|---|---|---|
| Catalyst | IV | (mins) | L* | a* | b* | L* | a* | b* |
| Example 1 | 0.62 | 90 | 74.11 | −1.55 | 6.83 | 72.83 | −1.93 | 8.70 |
| Example 5 | 0.62 | 71 | 74.44 | −2.79 | 10.43 | 75.07 | −2.04 | 10.23 |
| Sb₂O₃ | 0.6 | 122 | 59.58 | −0.83 | 2.69 | 61.25 | −0.94 | 3.64 |
| (Comp) | | | | | | | | |
| (270 ppm) | | | | | | | | |
| Example 10 | 0.62 | 108 | 70.10 | −2.48 | 14.55 | 72.55 | −2.40 | 17.16 |
| (comp) | | | | | | | | |
| (Av of 5 | | | | | | | | |
| runs) | | | | | | | | |

The results show that the catalysts of the invention give a very rapid polycondensation whilst the product polyester is significantly less yellow that the comparison titanium catalyst of Example 9. The melt stability, as evidenced by the colour change between polymer exiting the reactor after 5 and 15 minutes, is also very good using the catalysts of the invention. Compared with the antimony catalyst added at a relatively high concentration, the polycondensation time is much shorter using the catalysts of the invention and the resulting polymer is brighter (higher L* value) giving the polymer a desirable “sparkle”.

### EXAMPLE 13

### Hydrolysis Test

TABLE 2

| Catalyst | Precipitate | Colour |
|---|---|---|
| Example 1 | NO | Clear pale yellow solution |
| Example 3 | YES | Hazy yellow solution |
| Example 5 | NO | Clear pale yellow solution |
| Example 6 | YES | Hazy Yellow solution |
| Example 8 | YES | Hazy Yellow solution |
| Example 9 | YES | Hazy dark brown solution |
| Example 10 | NO | Clear yellow solution |

The hydrolytic stability of the titanium catalysts was determined by the following method. The required amount of the catalyst containing 350 ppm of Ti was added to 40 g of monoethylene glycol and 0.6 g of water (1.5%). The solution was thoroughly mixed and placed in a pressurized glass tube which was heated in an oven at 280° C. for 2 hours after which time the tube was removed and allowed to cool to room temperature. Any colour change or visible precipitation was recorded. The catalysts tested and the results are shown in Table 2 above.

### EXAMPLE 14-22

### Use of Co-Catalyst

Ethylene glycol (2.04 kg), isophthalic acid (125 g) and terephthalic acid (4.42 kg) were charged to a stirred, jacketed reactor. The reactor was heated to 226-252° C. at a pressure of 40 psi to initiate the first stage direct esterification (DE) process. Water was removed as it was formed with recirculation of the ethylene glycol. On completion of the DE reaction the contents of the reactor were allowed to reach atmospheric pressure before a vacuum was steadily applied. When the reactor was at atmospheric pressure phosphoric acid, the catalyst of Example 1, the co-catalyst (shown in Table 3&4), and an organic colour-management dye system (3 ppm Polysynthren™ Blue RBL and 2 ppm Polysynthren Red GFP, both available from Clariant) were added at about 5 minute intervals, if used, to allow for homogenisation. The amount of each additive in each polyester preparation is shown in Table 3 as ppm of the metal or phosphorus. The co-catalysts used were aqueous solutions of zinc acetate, magnesium acetate or calcium citrate respectively. The mixture was heated to 285±2° C. under vacuum to remove ethylene glycol and yield polyethylene terephthalate. The melt-polymerised polyester was discharged once a constant torque had been reached which indicated an IV of about 0.60 dl/g. The time for polycondensation (PC) and the intrinsic viscosity (IV) and colour values of the resulting polyesters are shown in Table 3.

500 g of the product polyester was crystallized in a rotary reactor in air at 160° C. for 30 minutes and then charged to a solid phase polymerisation reactor pre-heated to 210° C. SSP was carried out using a nitrogen sweep at a temperature of 210° C. The reaction was continued for 12 hours and samples were taken at the start of the reaction and at 2-hour intervals thereafter. Each sample was analysed for colour and IV by the methods described in Example 12. The IV was plotted versus time and the rate of solid phase polymerisation was calculated from change in IV per hour (dIV/dt(hr)). The IV rate as a % of the rate without the Zn co-catalyst and the colour of the resulting polyester after 12 hours of SSP are shown in Tables 3 & 4.

The results show that the SSP rate of polyester made using a catalyst system comprising a titanium catalyst according to the invention together with a zinc co-catalyst is surprisingly effective in producing good polymer which can be solid-phase polymerized in a relatively short time. In a comparison, a polyester was made according to the general method of Example 14, but using a catalyst system comprising 250 ppm of antimony (added as Sb₂O₃) with 80 ppm Zn co-catalyst together with the dye system and phosphoric acid. The resulting polymer showed a rate of SSP dIV/dt(hr) of 0.315 compared with 0.356 for the titanium based system containing a similar level of Zn (Example 15).

TABLE 3

| | Catalyst | | | | | Melt Reaction | | Colour (melt | | | Colour after | | |
|---|---|---|---|---|---|---|---|---|---|---|---|---|---|
| | system (all ppm) | | | | H₃PO₄ | DE | PC | polymerised) | | | 12 hours SSP | | |
| Example | Ti | Zn | Mg | Ca | ppm P | minutes | minutes | L | a | b | L | a | b |
| 14 | 12 | 0 | 0 | 0 | 10 | 99 | 217 | 57.45 | −2.56 | −4.12 | 78.9 | −1.4 | 0.4 |
| 15 | 12 | 110 | 0 | 0 | 17 | 85 | 75 | 55.25 | −2.97 | −3.95 | 78.9 | −3.25 | 1.9 |
| 16 | 12 | 80 | 0 | 0 | 17 | 87 | 97 | 53.69 | −1.94 | −5.18 | 76.89 | −2.67 | 0.03 |
| 17 | 12 | 60 | 0 | 0 | 17 | 93 | 104 | 58.05 | −2.26 | −4.85 | 80.95 | −2.56 | 0.49 |
| 18 | 12 | 50 | 0 | 0 | 17 | 90 | 119 | 57.83 | −3.87 | −4.76 | 81.2 | −2.9 | 0.25 |
| 19 | 12 | 40 | 0 | 0 | 17 | 85 | 134 | 57.69 | −2.15 | −5.33 | 81.15 | −2.05 | 0.15 |
| 20 | 12 | 30 | 0 | 0 | 17 | 84 | 149 | 55.99 | −3.12 | −1.65 | 79.5 | −1.9 | 1.9 |
| 21 | 12 | 0 | 120 | 0 | 10 | 89 | 112 | 55.3 | −3.98 | −0.98 | 82.5 | −2.25 | 5 |
| 22 | 12 | 0 | 0 | 180 | 10 | 84 | 130 | 56.19 | −2.16 | −3.86 | — | — | — |

TABLE 4

| | Catalyst | | | | | | | | | | | | |
|---|---|---|---|---|---|---|---|---|---|---|---|---|---|
| | system (all ppm) | | | | H₃PO₄ | IV at no of hours in SSP | | | | | | | % SSP |
| Example | Ti | Zn | Mg | Ca | ppm P | 0 | 2 | 4 | 6 | 8 | 10 | 12 | rate |
| 14 | 12 | 0 | 0 | 0 | 10 | 0.61 | 0.66 | 0.68 | 0.73 | 0.74 | 0.78 | 0.84 | 0 |
| 15 | 12 | 110 | 0 | 0 | 17 | 0.59 | 0.65 | 0.76 | 0.85 | 0.91 | 0.98 | 1.05 | 117 |
| 16 | 12 | 80 | 0 | 0 | 17 | 0.59 | 0.68 | 0.74 | 0.81 | 0.89 | 0.95 | 1.03 | 98 |
| 17 | 12 | 60 | 0 | 0 | 17 | 0.61 | 0.67 | 0.71 | 0.78 | 0.84 | 0.92 | 0.99 | 78 |
| 18 | 12 | 50 | 0 | 0 | 17 | 0.62 | 0.66 | 0.7 | 0.75 | 0.84 | 0.87 | 0.97 | 61 |
| 19 | 12 | 40 | 0 | 0 | 17 | 0.62 | 0.66 | 0.7 | 0.75 | 0.83 | 0.86 | 0.95 | 47 |
| 20 | 12 | 30 | 0 | 0 | 17 | 0.61 | 0.64 | 0.69 | 0.74 | 0.79 | 0.84 | 0.92 | 44 |
| 21 | 12 | 0 | 120 | 0 | 10 | 0.57 | 0.6 | 0.64 | 0.65 | 0.73 | 0.71 | 0.72 | −28 |

| | | | | | | | |
|---|---|---|---|---|---|---|---|
| 22 | 12 | 0 | 0 | 180 | 10 | 0.61 | hazy polymer - no SSP done |

1.-13. (canceled)

14. A catalyst suitable for use in an esterification reaction comprising the reaction product of
a) a compound of titanium, zirconium or hafnium
b) a 2-hydroxy carboxylic acid and
c) a quaternary ammonium compound selected from the group consisting of tetraethylammonium hydroxide and tetramethylammonium hydroxide.

15. A catalyst according to claim 14, wherein the compound of titanium, zirconium or hafnium is a compound of titanium.

16. A catalyst according to claim 14, wherein the compound of titanium, zirconium or hafnium is an alkoxide having the formula M(OR)₄ in which M is titanium, zirconium or hafnium and R is an alkyl group.

17. A catalyst according to claim 14, wherein the compound of titanium, zirconium or hafnium is a condensed alkoxide having the formula R¹O[M(OR¹)₂O]ₙR¹ in which R¹ represents an alkyl group, M represents titanium or zirconium and n is less than 20.

18. A catalyst according to claim 14, wherein the catalyst further comprises an alcohol.

19. A catalyst according to claim 18, wherein said alcohol contains at least two hydroxyl groups and comprises a dihydric alcohol selected from 1,2-ethanediol, 1,2-propanediol, 1,3-propanediol, 1,4-butane diol, diethylene glycol or a polyethylene glycol; or a polyhydric alcohol selected from glycerol, trimethylolpropane or pentaerythritol.

20. A catalyst according to claim 14, wherein the 2-hydroxy carboxylic acid is selected from the group consisting of lactic acid, citric acid, malic acid or tartaric acid.

21. A catalyst according to claim 14, wherein the molar ratio of 2-hydroxy carboxylic acid to titanium, zirconium or hafnium in the reaction product is 1 to 4 moles per mole of titanium, zirconium or hafnium.

22. A catalyst according to claim 14, wherein the amount of quaternary ammonium compound present is in the range 0.05 to 4 moles per mole of titanium, zirconium or hafnium.

23. A catalyst according to claim 14, further comprising a compound of zinc.

24. A process for the production of an ester, comprising reacting together an alcohol and at least one carboxylic acid, or an ester thereof, in the presence of a catalyst, said catalyst comprising the reaction product of
a) a compound of titanium, zirconium or hafnium
b) a 2-hydroxy carboxylic acid and
c) a quaternary ammonium compound selected from the group consisting of tetraethylammonium hydroxide and tetramethylammonium hydroxide.

25. A process for the production of a polyester comprising:
i) reacting together a polyhydroxy alcohol with at least one multifunctional carboxylic acid or an ester thereof to form a polyhydroxy ester of the multifunctional carboxylic acid
ii) polycondensing said polyhydroxy ester to form a polyester,
wherein at least one of steps i) and ii) is carried out in the presence of a catalyst, said catalyst comprising the reaction product of
a) a compound of titanium, zirconium or hafnium
b) a 2-hydroxy carboxylic acid and
c) a quaternary ammonium compound selected from the group consisting of tetraethylammonium hydroxide and tetramethylammonium hydroxide.

26. A process for the production of a polyester according to claim 25, comprising the steps of:
i) reacting together ethylene glycol with terephthalic acid or an ester thereof to form a bishydroxyethyl terephthalate,
ii) adding to the molten bishydroxyethyl terephthalate a stabiliser comprising a phosphorus-containing compound, a catalyst and a zinc compound, said catalyst comprising the reaction product of
a) a compound of titanium, zirconium or hafnium
b) a 2-hydroxy carboxylic acid and
c) a quaternary ammonium compound selected from the group consisting of tetraethylammonium hydroxide and tetramethylammonium hydroxide then
iii) polycondensing said bishydroxyethyl terephthalate to form polyethylene terephthalate.

27. A process according to claim 13, further comprising subjecting said polyethylene terephthalate to solid phase polymerisation.
